SEQUENCE LISTING (7 sequences)

SEQ ID NO: 1 - DNA, 3178 bp, Octopus minor
aatcgatatgtctcacactttgtggacgggattctttctctgtatactagggatagtttcccaagtgaaggcagataatgtggaatattttatcgcagcccaagaagttatctgggattatgccccgagtggaaatgatttggtcacaggaggcgatgattacaaaacattcttggagcacacttctaatagaattggaagtaagtacaagaaagtcatctatatgcagtatacagacttcacgtttactaaagaaataccgaagcctaaatcacttggtcttttgggacctattataagtggatctgttgggcaatcactccatgtttatttctttaacaatgccagtcgaccttataccattcacccacatggtcttttctacacaaaaggaaatgaaggagcattatatcgagatgagacaaagccatctgagaaagctgatgattctgttaaacctggacaaaccttcaactatacttggataatcgaaccctttgatggcccaacaaaagatgatgctgattgtctcccttggatgtatcactctcacattgaaccagttaaggatatgaatgctggccttatgggaatgttgattctctgcaaaaaatctaaaacatacaatcctgaaaacacccagcatttgttaatgaaagttatggatgaaaatttaagctggtaccttgaggaaaatattcaaatgttcataaacaatacggttgataaagaagatgaagatttccaggagagtaataaaatgcatgccttaaacggccttatgtatggcaacttggaattggaagcttgcattgctgataaagtcacttggtatttgatgggaatggggaatgaagtggacatacatactcttaatgttaatggtcattcctttgagtataatcatcacaggaaagagtccatcaacttgtacccagcagaattcagtgttgcagaaatggttatcgggagcgaaggatcatggctagtgaaatgtcatgttcatgaccattacgtgggtggcatggagaccttgttgaacgtgttctactgtaacaagaccaataactataatgtgggttctacgattataaattactttattacggctgaagaagtagaatggagttactcaagatctggcaggaatctctatgatggtggatctctcacagaacccgacagtgcttcagaagtatttttcaaaaaaggtaaacatagaattggtggaaaatacatgaaagctatgtattatcaatacactgatggtagttttacaacaaagatgtccagagggacagatgaagaacacctgggtaatttagggcctgtgattcgagctactgttggagacataatcaaagtagtattctttaataaggcatcacagaattattccattcatccatatggagtaagatataataaacctgatgagggtgctttctacactgatgggaaagataaaggtattgttccaccacaaacaagaaaaacttactactggtatatatcaagagatatgggacctcttgaaaaagatcctgattgtttgacgagtatgtatgcctcagaagtaaataccatgaaagacacatactctggccttgttggaccaatgcttatatgcaaaaagggtagtcttgatcggcatagaaaacagaaaagggtagataaagagttctttctcttatttatggtgactgatgaaaacaattcctggtacttgaaggagaacataaaaaagtacgctgaagatcctgaaggtgttgatttagaagatgaagattttatggaatcaaatttgatgcatggtatcaatggctacctttatgggaatctttatgaaccggaaatgtgcatgaatgataaaatcagctggcatatcatggctcttggcaacgaagtcgatttacacactgtttatttccatggaaacactttcactgttcatcaaaaccataaagatactttcagtgtacttccaggaatgttacaaacactagaaatgaaagctataaatcctggtacatgggctttggaatgtgtaaccaatgatcacttcaatgctggaacaaaggccatgtacaaagtacaacgctgtgggagacgacaaaaaactatatcacggaaagcgaaagttattgaatatttcattcaagctgaagaaattgaatgggattatgcaccaggtcataaagacctcatctataacaaatcccttgatgaccctgataatcctgggtatgcatttgttaaatcttcagattatctcattggaagcaaatatattaaagcagtttatcgaggttatacaaacgacagctttctaacacccctggaccacccagaatcatttggaattcttggacctctaataaaagctgaagtaggagatatcatatcagtgacctttaggaatttagcaagatataatttttcaattcttgctcatggagttctttatgaaaagtcaagtgaaggtacaaaatatgcagaccttaacgagaataaaggaaatgctgtcgaaaaggatgacatttacacatatacatggactgtaccaaagagttctggacctgggccaggggatccagattgtatcaattacgcatactattcagctgtaaacccatcacaagacacaaatgctgggttacttggacctctcgttatctgccgaaagaataaatcagactttaacaatacaatattcttcccacttttatttaatgtatttgatgaaaacgaaagtatatatcttgatgacaacattaagaaatacgcagaaaaggcaaataaagaagacgatgagtttattgagaacaacaaaaaccatgctattaatggttttctctttggaggtctgcaaggtctggatatgaataaacaggataaagtctactggtttttaataggaacaggaacagaagttgacatacattctattcatttccatggcaacacatttatccattatacagactacaaacacagaaaagatactttggatctgtggcccggttcatttaaaactatcttcatggttcctgaaaatccaggtaaatggttacttcactgccacgttgatgatcacatcatagggggtatgagtgcaatctatacagttaacgatatatcctaattccggacttgattgtaagaaaccctctcgagg

SEQ ID NO: 2 - protein, 1045 aa, Octopus minor
MSHTLWTGFFLCILGIVSQVKADNVEYFIAAQEVIWDYAPSGNDLVTGGDDYKTFLEHTSNRIGSKYKKVIYMQYTDFTFTKEIPKPKSLGLLGPIISGSVGQSLHVYFFNNASRPYTIHPHGLFYTKGNEGALYRDETKPSEKADDSVKPGQTFNYTWIIEPFDGPTKDDADCLPWMYHSHIEPVKDMNAGLMGMLILCKKSKTYNPENTQHLLMKVMDENLSWYLEENIQMFINNTVDKEDEDFQESNKMHALNGLMYGNLELEACIADKVTWYLMGMGNEVDIHTLNVNGHSFEYNHHRKESINLYPAEFSVAEMVIGSEGSWLVKCHVHDHYVGGMETLLNVFYCNKTNNYNVGSTIINYFITAEEVEWSYSRSGRNLYDGGSLTEPDSASEVFFKKGKHRIGGKYMKAMYYQYTDGSFTTKMSRGTDEEHLGNLGPVIRATVGDIIKVVFFNKASQNYSIHPYGVRYNKPDEGAFYTDGKDKGIVPPQTRKTYYWYISRDMGPLEKDPDCLTSMYASEVNTMKDTYSGLVGPMLICKKGSLDRHRKQKRVDKEFFLLFMVTDENNSWYLKENIKKYAEDPEGVDLEDEDFMESNLMHGINGYLYGNLYEPEMCMNDKISWHIMALGNEVDLHTVYFHGNTFTVHQNHKDTFSVLPGMLQTLEMKAINPGTWALECVTNDHFNAGTKAMYKVQRCGRRQKTISRKAKVIEYFIQAEEIEWDYAPGHKDLIYNKSLDDPDNPGYAFVKSSDYLIGSKYIKAVYRGYTNDSFLTPLDHPESFGILGPLIKAEVGDIISVTFRNLARYNFSILAHGVLYEKSSEGTKYADLNENKGNAVEKDDIYTYTWTVPKSSGPGPGDPDCINYAYYSAVNPSQDTNAGLLGPLVICRKNKSDFNNTIFFPLLFNVFDENESIYLDDNIKKYAEKANKEDDEFIENNKNHAINGFLFGGLQGLDMNKQDKVYWFLIGTGTEVDIHSIHFHGNTFIHYTDYKHRKDTLDLWPGSFKTIFMVPENPGKWLLHCHVDDHIIGGMSAIYTVNDIS

SEQ ID NO: 3 - protein, 21 aa, Artificial
  CP001
GSWLVKCHVHDHYVGGMETLL

SEQ ID NO: 4 - protein, 21 aa, Artificial
  CP002
GKWLLHCHVDDHIIGGMSAIY

SEQ ID NO: 5 - DNA, 28 bp, Artificial
  CP forward primer
aatcgatatgtctcacactttgtggacg

SEQ ID NO: 6 - protein, 28 aa, Artificial
  CP reverse primer
CCTCGAGAGGGTTTCTTACAATCAAGTC

SEQ ID NO: 7 - protein, 3 aa, Artificial
  CP002S
CPS

(19) United States
(12) Patent Application Publication
(10) Pub. No.: US 2023/0234993 A1
(43) Pub. Date: Jul. 27, 2023

(54) COMPOSITION FOR PREVENTING OR TREATING NEUROLOGICAL DISEASES RELATED TO COPPER METABOLISM, COMPRISING MULTI-COPPER OXIDASE PEPTIDE

(71) Applicant: NATIONAL MARINE BIODIVERSITY INSTITUTE OF KOREA, Janghang-eup Seocheon-gun Chungcheongnam (KR)

(72) Inventors: Seung Hyun JUNG, Seobuk-gu Cheonan-si, Chungcheongnam-do (KR); Seon Mi JO, Gunsan-si, Jeollabuk-do (KR); Jei Ha LEE, Seo-gu Daejeon (KR); Hye Suck AN, Janghang-eup Seocheon-gun Chungcheongnam (KR); Hea Bin KIM, Gunsan-si Jeollabuk-do (KR); Ye Rim SEO, Jung-gu Daejeon (KR)

(73) Assignee: NATIONAL MARINE BIODIVERSITY INSTITUTE OF KOREA, Janghang-eup Seocheon-gun Chungcheongnam (KR)

(21) Appl. No.: 18/010,139

(22) PCT Filed: Dec. 15, 2021
(86) PCT No.: PCT/KR2021/019085

§ 371 Date: Dec. 13, 2022

(30) Foreign Application Priority Data
Dec. 17, 2020 (KR) 10-2020-0177712

(51) Int. Cl. C07K 14/435; A61P 25/00

(52) U.S. Cl. CPC C07K 14/43504; A61P 25/00

(57) ABSTRACT

The present invention relates to a composition for preventing, ameliorating, or treating neurological diseases related to copper metabolism, wherein the composition comprises a multi-copper oxidase peptide. A multi-copper oxidase-derived peptide according to the present invention was found to be highly effective for treating myelination damage in the nervous system in a copper-induced neurological disease model, thus indicating that the multi-copper oxidase-derived peptide according to the present invention has a significant therapeutic effect on neurological diseases. Therefore, the multi-copper oxidase-derived peptide can be used in various ways in the field of preventing, ameliorating, and treating neurological diseases.

### TECHNICAL FIELD

The present invention relates to a composition for preventing, ameliorating, or treating neurological diseases related to copper metabolism including a multi-copper oxidase peptide.

### BACKGROUND ART

Ceruloplasmin is a major protein that transports copper in the blood and plays an important role in iron metabolism. The ceruloplasmin contains 6 copper atoms in a protein structure, and is known to be synthesized in the liver.

Meanwhile, copper is an essential catalytic and structural cofactor in many enzymes and is involved in major biological processes. Copper, an essential micronutrient, is balanced in the body by homeostasis of internalization and excretion. In fish, excess copper in the body damages the gills, liver, intestines, and nervous system. In humans, excess copper is a tumor promoter, stimulates the proliferation of cancer cells, and induces behavioral changes. In addition, excess copper in humans is known to cause Alzheimer's disease, and is also known to be associated with Wilson's disease, autism spectrum, and acute leukemia. As described above, excess copper in the body has toxicity, but the molecular mechanism related thereto is unknown.

### DISCLOSURE

### Technical Problem

Therefore, the present inventors prepared multi-copper oxidase-derived peptides CP001 and CP002; and CP002S, an active domain peptide derived from the CP002, confirmed a therapeutic effect of neurological diseases related to copper metabolism thereof, and completed the present invention.

Accordingly, an object of the present invention is to provide a peptide represented by amino acid sequences shown in SEQ ID NO: 3, 4, or 7.

Another object of the present invention is to provide a composition for preventing, ameliorating, or treating neurological diseases including one or more peptides selected from the group consisting of amino acid sequences shown in SEQ ID NOs: 3, 4, and 7.

Yet another object of the present invention is to provide a method for preventing or treating neurological diseases including administering one or more peptides selected from the group consisting of amino acid sequences shown in SEQ ID NOs: 3, 4, and 7 to a subject in need thereof.

### Technical Solution

In order to achieve the object, the present invention provides a peptide represented by an amino acid sequence shown in SEQ ID NO: 3.

The present invention also provides a peptide represented by an amino acid sequence shown in SEQ ID NO: 4.

The present invention also provides a peptide represented by an amino acid sequence shown in SEQ ID NO: 7.

The present invention also provides a pharmaceutical composition for preventing or treating neurological diseases including one or more peptides selected from the group consisting of amino acid sequences shown in SEQ ID NOs: 3, 4, and 7.

The present invention also provides a food composition for preventing or ameliorating neurological diseases including one or more peptides selected from the group consisting of amino acid sequences shown in SEQ ID NOs: 3, 4, and 7.

The present invention also provides a health functional food composition for preventing or ameliorating neurological diseases including one or more peptides selected from the group consisting of amino acid sequences shown in SEQ ID NOs: 3, 4, and 7.

The present invention also provides a method for preventing or treating neurological diseases including administering one or more peptides selected from the group consisting of amino acid sequences shown in SEQ ID NOs: 3, 4, and 7 to a subject in need thereof.

### Advantageous Effects

According to the present invention, it was confirmed that the multi-copper oxidase-derived peptide had an excellent effect on treating myelination damage in the nervous system in a copper-induced neurological disease model. This means that the multi-copper oxidase-derived peptide of the present invention has a significant therapeutic effect on neurological diseases. Therefore, the multi-copper oxidase-derived peptide can be used in various ways in the field of preventing, ameliorating, and treating neurological diseases.

### DESCRIPTION OF DRAWINGS

FIG. 1 is a diagram illustrating a result of confirming cDNA of obtained ceruloplasmin derived from Octopus minor through electrophoresis.

FIG. 2 is a diagram illustrating a hematological staining method and results of observing overexpression phenotypes of ceruloplasmin genes derived from Octopus minor through the hematological staining method.

FIG. 3 is a diagram illustrating results of searching for active domains of ceruloplasmin and results of analyzing homology with other organisms.

FIG. 4 is a diagram illustrating results of additionally analyzing optimal peptide sizes and activity information of peptides CP001, CP002, and CP002.

FIG. 5A is a diagram illustrating results of confirming the number of surviving subjects during treatment with multi-copper oxidase-derived peptides CP001 and CP002.

FIG. 5B is a diagram illustrating a result of calculating viability based on the result of confirming the number of surviving subjects of FIG. 4A.

FIG. 6A is a diagram illustrating results of confirming a concentration range in which multi-copper oxidase-derived peptides CP001 and CP002 are not toxic by confirming the number of surviving subjects.

FIG. 6B is a diagram illustrating a result of calculating viability based on the result of confirming the number of surviving subjects of FIG. 5A.

FIG. 7 is a diagram illustrating a result of confirming a therapeutic effect of a multi-copper oxidase-derived peptide on neurological diseases in a copper-induced neurological disease model through fluorescence microscopy.

FIG. 8 is a diagram illustrating a result of additionally analyzing a fluorescence microscope image according to an expression level of a fluorescent protein.

FIG. 9 is a diagram illustrating a result of examining a hatching rate according to CP002S treatment of the present invention under copper overexposure conditions.

FIG. 10A is a diagram illustrating results of observing that damage to myelinated cells in the nervous system is treated or prevented by a CP002S peptide in a copper-overexposed neurological disease model using a fluorescence microscope.

FIG. 10B is a diagram showing a graph prepared based on the results of observation under the fluorescence microscope of FIG. 10A.

FIG. 11A is a diagram illustrating a result of measuring a heart rate according to CP002S peptide treatment in a copper-overexposed nervous system model.

FIG. 11B is a diagram illustrating a result of calculating a heart rate inhibition rate compared to a normal group based on the heart rate measurement result of FIG. 11A.

### BEST MODE OF THE INVENTION

Hereinafter, the present invention will be described in detail.

According to an aspect of the present invention, there is provided a peptide represented by an amino acid sequence shown in SEQ ID NO: 3, 4, or 7.

In the present invention, the peptide refers to a linear molecule formed by binding amino acid residues with each other by peptide bonds. The peptide may be prepared according to a chemical synthesis method known in the art, and preferably prepared according to a solid-phase synthesis technique, but is not limited thereto.

The scope of the present invention includes functional equivalents of the peptide represented by the amino acid sequence shown in SEQ ID NO: 3, 4, or 7.

The functional equivalents refer to peptides which have sequence homology (that is, identity) of at least 80%, preferably 90%, more preferably 95% or more with the peptide represented by the amino acid shown in SEQ ID NO: 3, 4, or 7, and for example, sequence homology of 80%, 81%, 82%, 83%, 84%, 85%, 86%, 87%, 88%, 89%, 90%, 91%, 92%, 93%, 94%, 95%, 96%, 97%, 98%, 99%, and 100% as a result of addition, substitution, or deletion of amino acids, and exhibit substantially the same physiological activity as the peptide represented by the amino acid sequence shown in SEQ ID NO: 3, 4, or 7. In the present specification, the sequence homology and homogeneity are defined as a percentage of amino acid residues of a candidate sequence for the amino acid sequence shown in SEQ ID NO: 3, 4, or 7 after aligning the amino acid sequence shown in SEQ ID NO: 3, 4, or 7 and the candidate sequence and introducing gaps. If necessary, conservative substitution is not considered as part of sequence homogeneity in order to obtain the maximum percentage sequence homogeneity. An N-terminus, a C-terminus or internal extension, deletion, or insertion of the amino acid sequence represented by SEQ ID NO: 3, 4, or 7 is not construed as a sequence affecting sequence homogeneity or homology.

In addition, the sequence homogeneity may be determined by a general standard method used to compare similar portions of amino acid sequences of two polypeptides. A computer program such as BLAST or FASTA aligns the two polypeptides so as to optimally match respective amino acids (according to a full-length sequence of one or two sequences, or a predicted portion of one or two sequences). The program provides a default opening penalty and a default gap penalty and provides a scoring matrix such as PAM250 (standard scoring matrix) which may be used in association with the computer program. For example, the percentage homogeneity may be calculated as follows: The total number of identical matches is multiplied by 100 and then divided by a sum of the length of a longer sequence in a corresponding matched span and the number of gaps introduced into the longer sequence to align the two sequences.

The substantially homogeneous physiological activity means the activity of the peptide of the present invention, that is, the activity of preventing, ameliorating, or treating neurological diseases. The scope of the functional equivalents of the present invention includes derivatives in which some chemical structures of the peptide are modified while maintaining a basic skeleton and therapeutic activity against neurological diseases of the peptide represented by the amino acid sequence shown in SEQ ID NO: 3, 4, or 7. For example, the scope of the functional equivalents includes structural modifications for changing the stability, storage, volatility, solubility, or the like of the peptide.

The peptide represented by the amino acid sequence shown in SEQ ID NO: 3 or 4 according to the present invention is derived from ceruloplasmin-derived multi-copper oxidase of Octopus minor, and was referred to as ‘CP001’ and ‘CP002’, respectively.

The peptide represented by the amino acid sequence shown in SEQ ID NO: 7 according to the present invention is determined based on a result of predicting the optimal peptide size and activity information of CP002 represented by the amino acid sequence shown in SEQ ID NO: 4 through ExPASy prosite, and consists of 12 amino acids derived from CP002 (1024 to 1035 positions of SEQ ID NO: 2). The present inventors named the peptide represented by the amino acid sequence shown in SEQ ID NO: 7 as ‘CP002S’.

In an embodiment of the present invention, the peptide represented by the amino acid sequence shown in SEQ ID NO: 3, 4, or 7 is derived from preferably ceruloplasmin, more preferably ceruloplasmin of Octopus minor, but is not limited thereto.

It was confirmed that the peptide represented by the amino acid sequence shown in SEQ ID NO: 3, 4, or 7 according to the present invention had not only no developmental toxicity, but also an effect of treating myelination damage in the nervous system in a copper-induced neurological disease model. Accordingly, the peptide may be used in various ways in the field of preventing, ameliorating, and treating neurological diseases.

According to another aspect of the present invention, there is provided a composition for preventing, ameliorating, or treating neurological diseases including one or more peptides selected from the group consisting of amino acid sequences shown in SEQ ID NOs: 3, 4, and 7. The composition for preventing, ameliorating, or treating neurological diseases according to the present invention may be a pharmaceutical composition, a food composition, or a health functional food composition.

In an embodiment of the present invention, the concentration of the peptide is preferably 0.01 to 20 μM. When the concentration of the peptide included in the composition of the present invention is 0.01 μM, it is not preferred in that the therapeutic effect on neurological diseases is insignificant, and when the concentration of the peptide exceeds 20 μM, it is not preferred in that the peptide exhibits developmental toxicity.

In an embodiment of the present invention, the neurological diseases are preferably demyelinating neurological diseases.

In the present invention, the demyelinating neurological diseases refer to diseases occurring by myelin damage caused by an immunological mechanism or metabolic abnormality. The myelin damage occurs when the myelin covering the axon is eliminated by pathological changes in the myelin itself or the oligodendrocytes (central nervous system) or schwann cells (peripheral nervous system) generating the myelin.

In a preferred embodiment of the present invention, the demyelinating neurological diseases are preferably one or more selected from the group consisting of multiple sclerosis, ophthalmoneuromyelitis, and acute demyelinating encephalomyelitis, but are not limited thereto.

In an embodiment of the present invention, the neurological diseases are preferably neurological diseases induced by copper metabolism disorders.

The composition for preventing, ameliorating, or treating neurological diseases of the present invention may further include one or more known active ingredients having the same activity together with the peptide represented by the amino acid sequence shown in SEQ ID NO: 3, 4, or 7.

In a preferred embodiment of the present invention, the neurological diseases induced by the copper metabolic disorders are preferably one or more selected from the group consisting of Parkinson's disease, Alzheimer's disease, amyotrophic lateral sclerosis, Huntington's disease, Menkes disease, Wilson's disease, Prion's disease, occipital horn syndrome, Aceruloplasminemia, abnormal sensory symptoms, movement disorders, and optic neuritis, but are not limited thereto.

In a more preferred embodiment of the present invention, the neurological diseases may be neurological diseases caused by myelination defects induced by copper metabolism disorders, and for example, may be one or more selected from the group consisting of multiple sclerosis, Parkinson's disease, Alzheimer's disease, Menkes disease, and Wilson's disease. The above-mentioned neurological diseases are diseases proven association with copper overexposure, and a demyelination defect repair effect.

The composition of the present invention may be a pharmaceutical composition, a food composition, or a health functional food composition.

When the composition of the present invention is a pharmaceutical composition for preventing or treating neurological diseases, the pharmaceutical composition of the present invention may further include a pharmaceutically acceptable additive. At this time, the pharmaceutically acceptable additive may include starch, gelatinized starch, microcrystalline cellulose, lactose, povidone, colloidal silicon dioxide, calcium hydrogen phosphate, lactose, mannitol, syrup, gum arabic, pregelatinized starch, corn starch, powdered cellulose, hydroxypropyl cellulose, Opadry, sodium starch glycolate, carnauba wax, synthetic aluminum silicate, stearic acid, magnesium stearate, aluminum stearate, calcium stearate, white sugar, etc. The pharmaceutically acceptable additive according to the present invention is preferably included in an amount of 0.1 to 90 parts by weight based on the composition, but is not limited thereto.

The pharmaceutical composition of the present invention may be administered in various oral or parenteral formulations during actual clinical administration, but may be prepared using commonly used diluents or excipients, such as fillers, extenders, binders, wetting agents, disintegrants, surfactants, etc., for formulations. Suitable formulations known in the art are preferably used with formulations disclosed in the literature (Remington's Pharmaceutical Science, recently, Mack Publishing Company, Easton Pa.).

Solid formulations for the oral administration include tablets, pills, powders, granules, capsules, and the like, and the solid formulations may be prepared by mixing at least one excipient, for example, starch, calcium carbonate, sucrose or lactose, gelatin, and the like. Further, lubricants such as magnesium stearate and talc may be used in addition to simple excipients. In addition, liquid formulations for the oral administration may correspond to a suspension, an oral liquid, an emulsion, a syrup, and the like, and may include various excipients, for example, a wetting agent, a sweetener, an aromatic agent, a preserving agent, and the like, in addition to water and liquid paraffin which are commonly used as simple diluents.

The formulations for the parenteral administration include a sterile aqueous solution, a non-aqueous solution, a suspension, an emulsion, a lyophilizing agent, and a suppository. As the non-aqueous solution and the suspension, propylene glycol, polyethylene glycol, vegetable oil such as olive oil, injectable ester such as ethyl oleate, and the like may be used. As a base compound of the suppository, witepsol, macrogol, tween 61, cacao butter, laurinum, glycerogelatin, and the like may be used.

The dosage of the pharmaceutical composition of the present invention may vary depending on a formulation method, an administration method, an administration time, and/or a route of administration of the pharmaceutical composition. In addition, the dosage may vary depending on various factors including the type and degree of a response to be achieved by administration of the pharmaceutical composition, the type, age, weight, general health status, symptoms or severity of a disease, sex, diet, and excretion of a subject to be administered, drugs used simultaneously or separately for the corresponding subject, other composition ingredients, and the like, and similar factors well-known in the field of medicine. A person of ordinary skill in the art may easily determine and prescribe an effective dosage for a desired treatment.

The dosage of the pharmaceutical composition of the present invention may be administered at a concentration of, for example, preferably 0.05 to 5 mg/kg, more preferably 0.1 to 0.4 mg/kg, much more preferably 0.2 to 0.35 mg/kg, even more preferably 0.25 mg/kg, but the dosage does not limit the scope of the present invention in any way.

The administration route and the administration method of the pharmaceutical composition of the present invention may be independent of each other, and the method is not particularly limited, and may be any administration route and administration method so long as the pharmaceutical composition may reach a target site.

The pharmaceutical composition may be administered by oral administration or parenteral administration. The parenteral administration method includes, for example, intravenous administration, intraperitoneal administration, intramuscular administration, transdermal administration, or subcutaneous administration.

The pharmaceutical composition of the present invention may be used alone or in combination with methods using surgery, radiation therapy, hormone therapy, chemotherapy, and biological response modifiers for the prevention or treatment of neurological diseases.

The composition of the present invention may be a food composition or a health functional food composition for preventing or ameliorating neurological diseases.

In the present invention, the food refers to food having bioregulatory functions, such as prevention and amelioration of the disease, biophylaxis, immunity, recovery from illness, aging suppression, etc., and needs to be harmless to the human body when taken for a long period of time.

When the food composition of the present invention is a food additive, the active ingredient, the peptide represented by the amino acid sequence shown in SEQ ID NO: 3, 4, or 7, may be added as it is or used with other foods or food ingredients, and appropriately may be used according to a conventional method. The mixed amount of the active ingredients may be suitably determined according to the purpose of use (prevention, health, or therapeutic treatment). In general, the peptide, which is the active ingredient of the present invention, is added in an amount of 15 wt % or less, preferably 10 wt % or less, based on the raw material when preparing foods or beverages. However, in the case of long-term intake for the purpose of health and hygiene or for the purpose of health control, the amount of the peptide may be equal to or greater than the range, and there is no problem in terms of safety, so that the active ingredients may be used even in an amount above the range.

The kind of food is not particularly limited. Examples of the food which may be added with the materials include meat, sausages, bread, chocolate, candies, snacks, confectionery, pizza, ramen, other noodles, gums, dairy products including ice cream, various soups, beverages, tea, drinks, alcohol drinks, vitamin complex, and the like, and include all health functional foods in an accepted meaning.

The health functional food composition according to the present invention may be in various forms such as health drinks. When the health functional food composition according to the present invention is in a health drink form, various flavoring agents or natural carbohydrates may be added as an additional ingredient like general drinks. As the above-mentioned natural carbohydrates, monosaccharides such as glucose and fructose, disaccharides such as maltose and sucrose, natural sweeteners such as dextrin and cyclodextrin, synthetic sweeteners such as saccharin and aspartame, and the like may be used. A ratio of the natural carbohydrates may be generally about 0.01 to 10 g, preferably about 0.01 to 0.1 g per 100 ml of the composition of the present invention.

In addition, the food composition or the health functional food composition of the present invention may include various nutrients, vitamins, electrolytes, flavoring agents, coloring agents, pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, or the like. In addition, the composition of the present invention may include pulp for preparing natural fruit juice, fruit juice beverage, or vegetable beverage. These ingredients may be used independently or in combination. Although the ratio of the additives is not greatly important, generally, the ratio thereof is selected in a range of 0.01 to 0.1 part by weight per 100 parts by weight of the composition of the present invention.

According to yet another aspect of the present invention, there is provided a method for preventing or treating neurological diseases including administering one or more peptides selected from the group consisting of amino acid sequences shown in SEQ ID NOs: 3, 4, and 7 to a subject in need thereof.

In an embodiment of the present invention, the subject is a subject expected to develop neurological diseases; a subject having neurological diseases; or a subject completely cured of neurological diseases, but is not limited thereto.

Duplicated contents are omitted in consideration of the complexity of the present specification, and terms not defined otherwise in the present specification have the meanings commonly used in the art to which the present invention pertains.

### Modes of the Invention

Hereinafter, the present invention will be described in more detail through Examples. These Examples are just illustrative of the present invention, and it will be apparent to those skilled in the art that it is not interpreted that the scope of the present invention is limited to these Examples.

### Example 1. Isolation of Ceruloplasmin Derived from Octopus Minor

1-1. Obtaining of cDNA of Ceruloplasmin Derived from Octopus Minor

In order to isolate a ceruloplasmin gene encoding multicopper oxidase 1 (MCO1) from Octopus minor, samples for each part of adult Octopus minor and samples for each development stage of Octopus minor were prepared. Total RNA was extracted from the prepared sample. After the extracted total RNA was quantified, a cDNA library was prepared using an oligo dT primer and reverse transcriptase (Superscript IV first-strand synthesis system, Invitrogen). The cDNA library was used as a template for gene isolation.

Primers for amplifying ceruloplasmin were prepared from the cDNA library, and specific sequences were shown in Table 1.

TABLE 1

| | | | Size (bp) of |
|---|---|---|---|
| | Forward primer | Reverse primer | amplification |
| Gene | (5’ to 3’) | (5’ to 3’) | product |
| Ceruloplasmin | AATCGATATGTCTC | CCTCGAGAGGGTTT | 3178 |
| | ACACTTTGTGGAC | CTTACAATCAAGTC | |
| | G (SEQ ID NO: 5) | (SEQ ID NO: 6) | |

In order to isolate the ceruloplasmin gene, PCR was performed with an AccuPower PCR Premix & Master Mix kit (Bioneer). Specifically, 3 μl of each of the forward and reverse primers in Table 1 and 1 μl of the cDNA library were prepared, and a final volume was adjusted to 30 μl with tertiary sterile distilled water. PCR was performed under conditions of Table 2.

TABLE 2

| Repetition | | | |
|---|---|---|---|
| number | Condition | Temperature (° C.) | Time (min) |
| 1 | Pre-denaturation | 95 | 5 |
| 33 | Denaturation | 95 | 0.5 |
| | Annealing | 56 | 0.5 |
| | Extension | 72 | 1.5 |
| 1 | Post-extension | 72 | 7 |

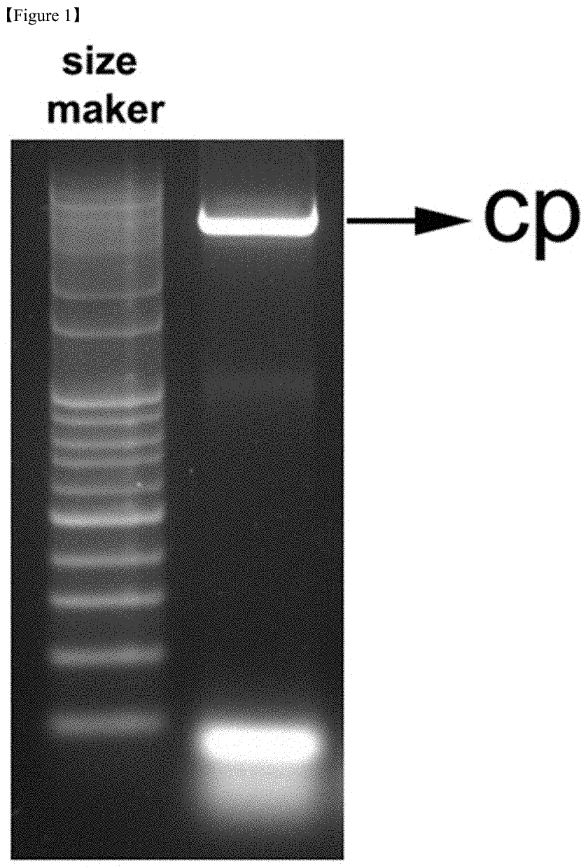

The amplification product obtained through PCR was confirmed through electrophoresis using an agarose gel. The electrophoresis result was illustrated in FIG. 1.

As illustrated in FIG. 1, the amplification product identified a band at an expected position (i.e., 3178 bp).

For subsequent experiments, the band portion of the agarose gel was separated with a sterilized knife, and DNA was purified using a gel extraction kit (Qiagen).

1-2. Nucleotide Sequence and Amino Acid Sequence Analysis of Ceruloplasmin Derived from Octopus Minor

An experiment was performed to confirm a nucleotide sequence of purified DNA. Specifically, pGEM T-easy vector (Promega) was used for ligation at 4° C. for 12 hours. The ligated DNA was inserted into a competent cell (DH5a, RBC) by a transformation method, and clones into which target DNA was inserted were selected by white colony and colony PCR methods. The selected clones were cultured in an LB broth. After taking 8 ml of the culture medium, the DNA inserted into a cloning vector was obtained using a Plasmid DNA miniprep kit (Qiagen). Analysis of the sequence of the obtained DNA was requested and performed by Macrogen, Inc. (Korea).

As a result of the sequence analysis, it was confirmed that the nucleotide sequence of the obtained DNA (i.e., ceruloplasmin gene) was represented by SEQ ID NO: 1.

In addition, it was confirmed that NCBI blastx and blastp were aligned with a reference and model organisms database, and the ceruloplasmin gene (SEQ ID NO: 1) identified above was translated into the amino acid sequence shown in SEQ ID NO: 2.

### Example 2. Confirmation of Abnormal Blood Deposition in Tissue Through Hematological Staining Method

An overexpression phenotype of a ceruloplasmin gene derived from Octopus minor was observed in zebrafish, a model animal. Specifically, the ceruloplasmin gene (SEQ ID NO: 1) derived from Octopus minor obtained in Example 1 was treated with restriction enzymes Cla 1 and Xho 1, and inserted into a pCS2+ expression vector. A restriction enzyme Sac II was treated to the pCS2+ expression vector into which the ceruloplasmin gene was inserted, to prepare linear DNA. An mRNA using the linear DNA as a template was synthesized by an in vitro transcription (Sp6, Invitrogen) method.

The synthesized ceruloplasmin mRNA was isolated by phenol/chloroform purification and then stored at −70° C.

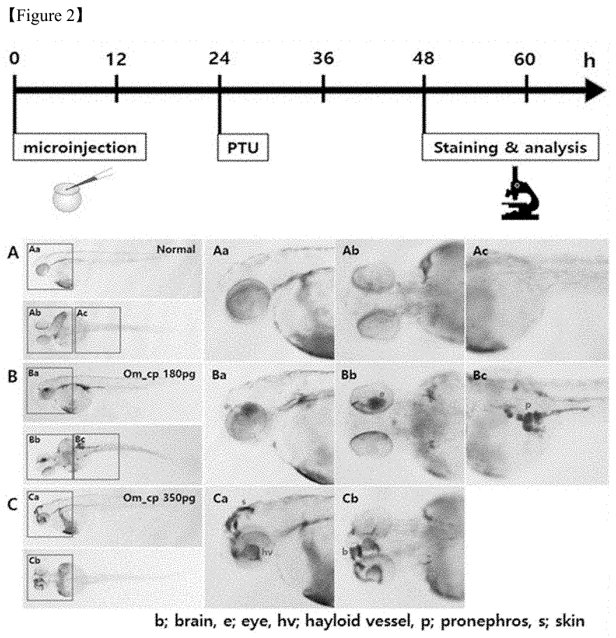

After obtaining fertilized eggs by mating wild-type zebrafish, embryos at 1 to 4 cell stages were arranged on an injection plate. After loading the synthesized ceruloplasmin mRNA in a microglass tube with a microloder tip, a predetermined concentration (180 pg or 350 pg) thereof was injected into the embryo using a microinjector, a picopump, and a microscope. Subjects injected with the ceruloplasmin mRNA were cultured in an incubator, and after 48 hours of injection, dark-treated with a hematological staining reagent (o-dianisidine (0.6 mg/ml), 0.01 M sodium acetate (pH 4.5), 0.65% H₂O₂, 40% ethanol) for 30 minutes, and then washed. After washing, a fixing solution (4% paraformaldehyde) was treated to the subject injected with the ceruloplasmin mRNA, and reacted for at least 1 hour. The subjects injected with the immobilized ceruloplasmin mRNA were placed in 80% glycerol/20% PBS, and photographed under a stereoscopic microscope. The results of microscopic observation were illustrated in FIG. 2.

As illustrated in FIG. 2, abnormal blood deposition in tissue was observed in the subjects injected with the ceruloplasmin mRNA due to the in vivo overexpression of the ceruloplasmin protein compared to a normal group.

### Example 3. Search of Active Domain of Ceruloplasmin Derived from Octopus Minor

Based on the amino acid sequence (SEQ ID NO: 2) of ceruloplasmin derived from Octopus minor confirmed in Example 1, active domains of ceruloplasmin were searched. The active domain search was performed using ExPASy prosite (https://prosite.expasy.org/) and MOTIF Search (https://www.genome.jp/tools/motif/).

Based on information on the searched active domains of ceruloplasmin, the homology between the active domains of ceruloplasmin derived from Octopus minor and active domains derived from human, mouse, and fish were analyzed. The homology analysis was performed using a ClustalW program (https://www.genome.jp/tools-bin/clustalw).

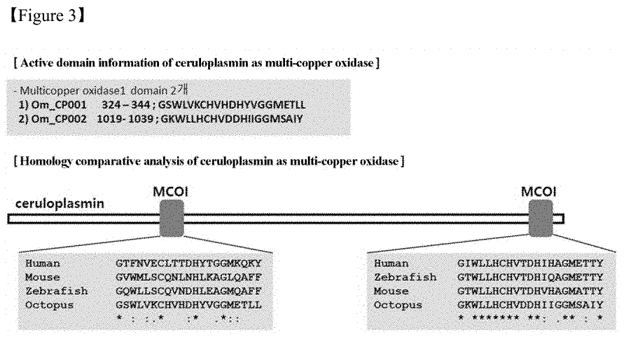

The results of homology analysis of the searched active domains of ceruloplasmin with other organisms were illustrated in FIG. 3.

As illustrated in FIG. 3, it was confirmed that the searched active domains of ceruloplasmin were amino acids 324 to 344; and amino acids 1019 to 1039 of ceruloplasmin. The two active domains were named CP001 (324 to 344, SEQ ID NO: 3) and CP002 (1019 to 1039, SEQ ID NO: 4), respectively. It was confirmed that the CP001 and CP002 sites were homologous to other organisms (human, mouse, and fish), but some amino acids were different.

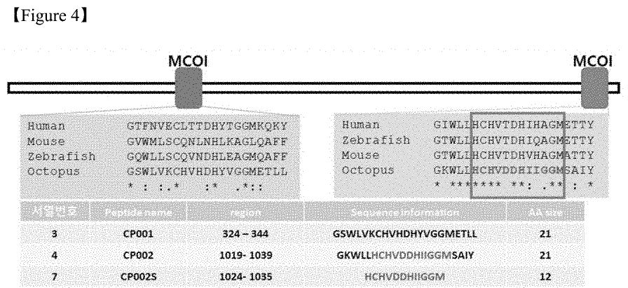

The optimal peptide sizes and activity information of the peptides CP001 and CP002 were further analyzed. The analysis was performed using an active domain search site [ExPASy prosite (https://prosite.expasy.org/), and MOTIF Search (https://www.genome.jp/tools/motif/)]. The results of analyzing the optimal peptide sizes and the activity information were illustrated in FIG. 4.

As illustrated in FIG. 4, as a result of predicting the activity in ExPASy prosite, when CP001 was reduced from the length of 21 amino acids, it was impossible to predict the activity as multi-copper oxidase, and the activity of CP002 was predicted even with a length of up to 12 amino acids. As a result of predicting the activity in the MOTIF Search site, as a result of reducing the peptide lengths of CP001 and CP002, it was impossible to predict the activity of the two peptides. Therefore, based on the optimal peptide sizes and activity prediction results using the activity information analysis program (ExPASy prosite), 12 amino acids (1024 to 1035 of SEQ ID NO: 2) derived from CP002 were requested and synthesized by a biostem company. The 12 peptides derived from CP002 were named CP002S and represented by the amino acid sequence shown in SEQ ID NO: 7.

### Example 4. Developmental Toxicity Analysis of Multi-Copper Oxidase-Derived Peptides

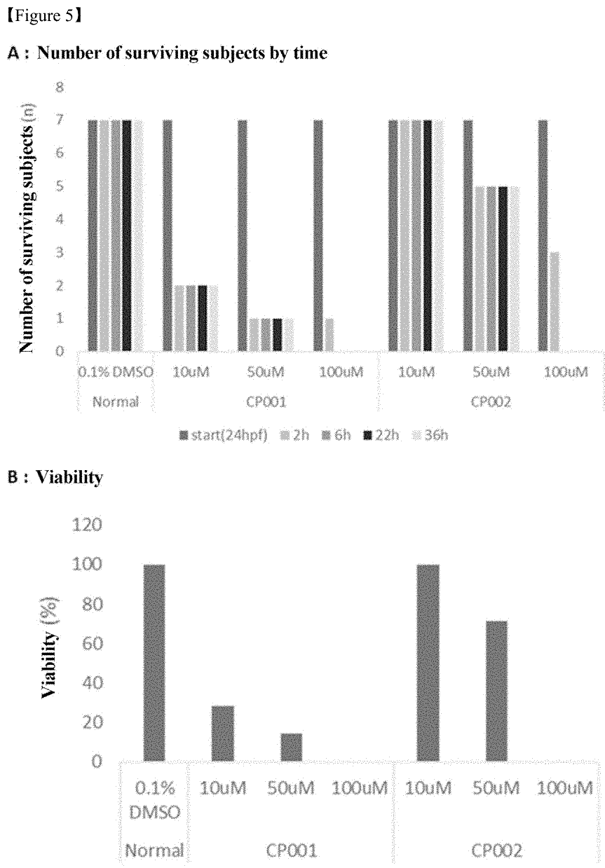

The developmental toxicity of CP001 (SEQ ID NO: 3) and CP002 (SEQ ID NO: 4), which were multi-copper oxidase-derived peptides, was analyzed. Specifically, fertilized eggs were obtained by mating wild-type zebrafish. After the obtained fertilized eggs were maintained in an incubator for 24 hours, 7 subjects were dispensed into each well of a 24-well plate. The dispensed subjects were treated with CP001 and CP002 at various concentrations (10, 50, and 100 μM). After treatment with the CP001 and CP002, the number of surviving subjects at 2, 6, 22, and 36 hours was confirmed using a microscope, and the viability was calculated based on the results. The control group of the experiment was treated with 0.1% DMSO. The result of confirming the number of surviving subjects was illustrated in FIG. 5A and the calculation result of the viability was illustrated in FIG. 5B.

As illustrated in FIGS. 5A and 5B, developmental toxicity was confirmed in a group treated with CP001 at a concentration of 10 to 100 μM. In addition, the developmental toxicity was not shown in the group treated with CP002 at a concentration of 10 μM, but the developmental toxicity was confirmed in a group treated with a concentration of 50 to 100 μM.

Accordingly, the concentration range in which CP001 and CP002 did not exhibit the developmental toxicity was further examined. The experiment was performed in the same method as described above, and the treatment concentrations of CP001 and CP002 were as follows.
CP001: 1 μM, 4 μM
CP002: 5 μM, 10 μM

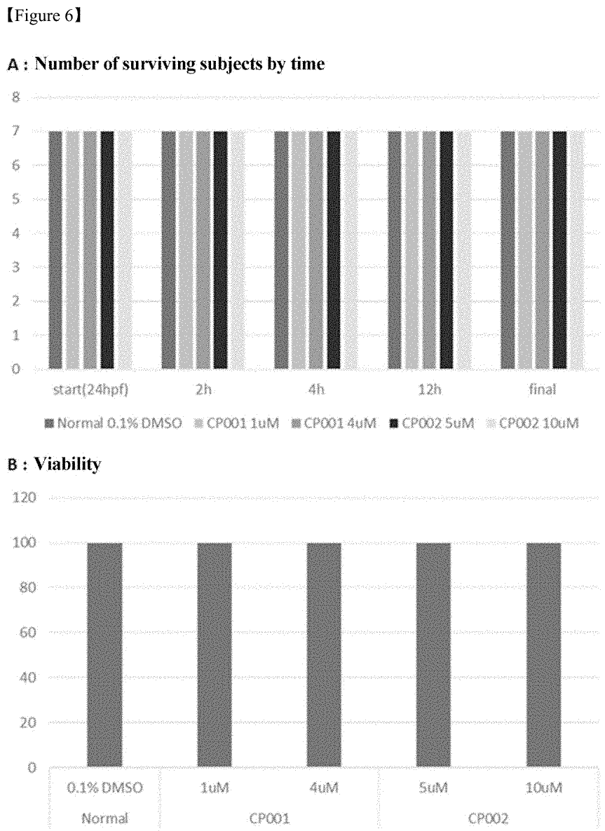

The results of further examining the developmental toxicity of CP001 and CP002 were illustrated in FIG. 6 (A: number of surviving subjects by time, B: viability).

As illustrated in FIGS. 6A and 6B, it was confirmed that in a group treated with CP001 at a concentration of 1 or 4 μM; and a group treated with CP002 at a concentration of 5 or 10 μM, the number of surviving subjects and the viability were high (i.e., there was no developmental toxicity). In the case of CP001, the developmental toxicity was not confirmed even at a concentration of 5 μM, and thus, future experiments were performed at a concentration of up to 5 μM.

### Example 5. Examination of Therapeutic Effect of Multi-Copper Oxidase-Derived Peptides on Neurological Diseases Related to Copper Metabolism

The therapeutic effect of multicopper oxidase-derived peptides CP001 (SEQ ID NO: 3) and CP002 (SEQ ID NO: 4) were examined in a neurological disease model caused by copper overexposure.

5-1. Construction of Neurological Disease Model Caused by Copper Overexposure

A zebrafish transformant (mbp; mGFP transgenic) expressing a fluorescent protein GFP in the myelin surrounding the nervous system was prepared. The developmental process of fertilized eggs obtained by mating the male and female zebrafish transformants was safely observed and maintained in an incubator. After 24 hours of fertilization, normally grown embryos were dispensed into a 24-well plate by 13 subjects. The dispensed embryos were divided into a normal group (0.1% DMSO); a positive control treated with copper alone (4 μM CuSO₄); a copper and CP001 treated group (1 and 5 μM); and a copper and CP002 treated group (5 and 10 μM), and treated with copper and multi-copper oxidase-derived peptides according to the conditions of each group. In addition, in order to observe the expression of the fluorescent protein to the inside of the tissue, 1-phenyl 2-thiourea (PTU), a pigmentation inhibitor, was used as a basic culture medium for all the experimental groups.

5-2. Examination of Therapeutic Effect of Multi-Copper Oxidase-Derived Peptides in Neurological Disease Model

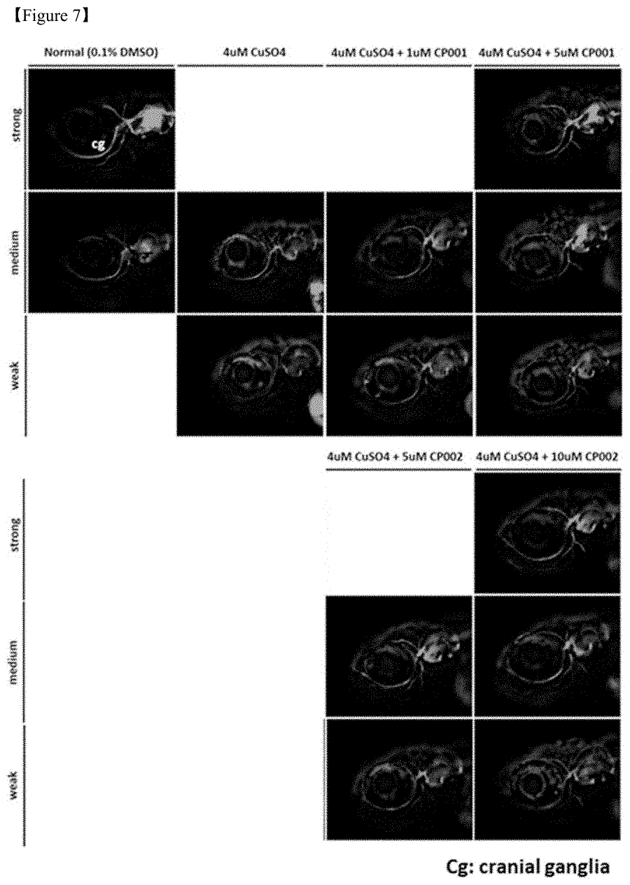

After 5 days of treatment with copper and multi-copper oxidase-derived peptides, a neurological disease model was anesthetized with tricaine (ethyl 3-aminobenzoate methanesulfonate salt, 4 g/L. pH 7.0). Anesthetized subjects were placed on a 3% methylcellulose solution and observed under a fluorescence microscope. The results of observation under a fluorescence microscope were shown in FIG. 7.

As illustrated in FIG. 7, it was confirmed that in a normal group (normal), the fluorescent proteins were normally expressed in the myelinated cells of the nervous system. On the other hand, it was confirmed that a positive control group treated with copper (4 μM CuSO₄) had a defect in myelination. However, it was confirmed that the multi-copper oxidase peptides CP001 and CP002 treated groups partially recovered the myelination.

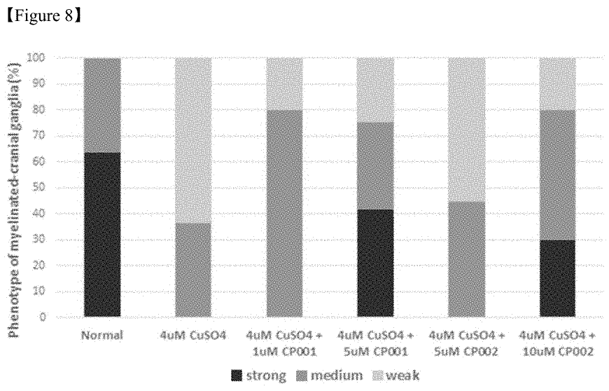

In addition, additional analysis was performed according to expression levels of the fluorescent protein divided into strong, medium, and weak in cranial ganglia. The analysis results according to the fluorescent protein expression level were illustrated in FIG. 8.

As illustrated in FIG. 8, it was confirmed that a normal group (normal) had strong 64% and medium 36%, and the fluorescent protein expression was normal in the cranial ganglia. On the other hand, it was confirmed that a positive control group treated with copper (4 μM CuSO₄) had 36% medium and weak 64%, and the expression of the fluorescent protein was reduced compared to the normal group. This means that copper inhibited myelination (demyelination), that is, induced neurological diseases.

A copper and multi-copper oxidase peptide CP001-treated group (1 μM) had 80% medium and weak 20%, and thus, it was confirmed that the expression of the fluorescent protein was increased compared to the positive control group. The group treated with 5 μM of CP001 also showed the same trend (strong 42%, medium 33%, and weak 25%).

A copper and multi-copper oxidase peptide CP002-treated group (5 μM) had 44% medium and weak 56%, and thus, it was confirmed that the expression of the fluorescent protein was increased compared to the positive control group. The group treated with 10 μM of CP002 also showed the same trend as described above (strong 30%, medium 50%, and weak 20%).

The results show that copper induces demyelination, and multi-copper oxidase peptides CP001 and CP002 treat myelination damage in the nervous system in a copper-induced neurological disease model.

Meanwhile, a representative neurological disease caused by the demyelination is multiple sclerosis. In the multiple sclerosis, inflammatory activity due to immune system damage is abnormally increased, and axonal or synaptic degeneration, and death phenomena of synapses and neurons and oligodendrocytes were observed. Such multiple sclerosis treatment is impossible only by simply prescribing an immunomodulatory agent, and drugs for neuroprotection and damage recovery, and the like are required (Pablo Villoslada. 2016. BioMed central). Therefore, the multi-copper oxidase peptides CP001 and CP002, which have been proven to be effective in treating myelination damage in the nervous system, may be applied to novel therapeutic agents and treatment methods for neurological diseases caused by demyelination.

### Example 6. Comparative Analysis of Treatment Concentration of CP002S in Embryos of Model Animal

6-1. Analysis of Viability According to Treatment Time of CP002 and CP002S

The developmental toxicity of CP002S treatment was analyzed in the embryos of zebrafish exposed to copper. Specifically, the development process of fertilized eggs obtained by mating male and female wild-type zebrafish was safely maintained in an incubator (28.5° C.). At 24 hours after fertilization, normally grown embryos were dispensed into a 24-well plate by 13 subjects and maintained under copper overexposure conditions. The dispensed subjects were treated with CP002 (5, 10 μM) or CP002S (5, 10 μM). With reference to the results of confirming the developmental toxicity of treatment of CP002 alone in the previous studies, the experimental concentration range for CP002S was selected as 5 and 10 μM. After treatment with CP002S, the number of surviving subjects at 0, 4, 7, 12, and 24 (for 24, 28, 31, 36, and 48 hours of the development) was confirmed using a microscope, and the viability was calculated based on the results. A negative control group in the experiment was a 0.1% DMSO treated group (normal group) and a copper overexposed group, and a positive control group was a CP002 treated group; a DPA (penicillamine/D-Penicillamine) treated group; and a TETA (trientine/Trientine) treated group. The DPA and TETA were drugs that have been used as therapeutic agents for Wilson's disease and copper overexposure in the related art.

As a result of analyzing the viability according to CP002S treatment, all groups showed a decrease in viability of about 15% to 35%. This is considered to be a phenomenon caused by a difference by subject rather than induction of serious toxicity to the treated drugs, and it is determined not to cause the developmental toxicity at the treatment concentration of each drug.

### Example 7. Examination of Hatching Rate According to Treatment of CP002 and CP002S Under Copper Overexposure Conditions

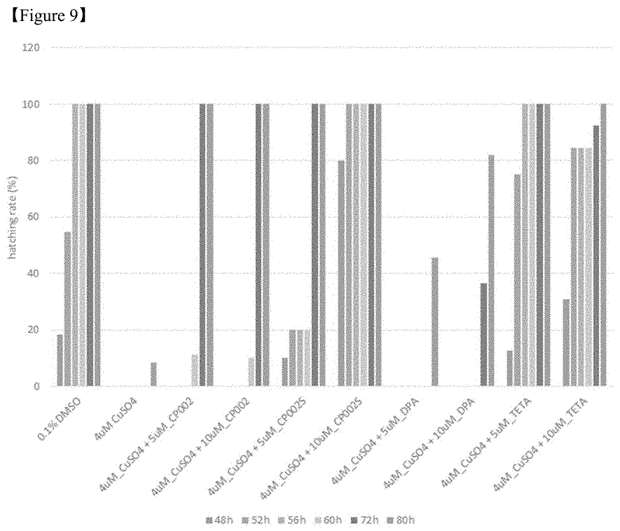

Through hatching rate examination, the activities of CP002 and CP002S were analyzed in zebrafish embryos exposed to copper. Specifically, the development process of fertilized eggs obtained by mating male and female wild-type zebrafish was safely maintained in an incubator. At 24 hours after fertilization, normally grown embryos were dispensed into a 24-well plate by 13 subjects and maintained under copper overexposure conditions. The dispensed subjects were treated with CP002 (5, 10 μM), CP002S (5, 10 μM), DPA (5, 10 μM), or TETA (5, 10 μM). From 48 hours to 52, 56, 60, 72, and 80 hours after fertilization (24 hours after treatment) when hatching started, the number of hatched subjects in each group was examined. A negative control group in the experiment was a 0.1% DMSO treated group (normal group) and a copper overexposed group (4 μM CuSO₄), and a positive control group was a CP002 treated group; a DPA (penicillamine/D-Penicillamine) treated group; and a TETA (trientine/Trientine) treated group. The DPA and TETA were drugs that have been used as therapeutic agents for Wilson's disease and copper overexposure in the related art. The results of examining the hatching rate were illustrated in FIG. 9.

As illustrated in FIG. 9, in the case of a normal group (0.1% DMSO), hatching rates of 18% at 48 hours, 54.5% at 52 hours, and 100% at 56 hours after fertilization when hatching started were observed. In the copper overexposure (4 μM CuSO₄) group, the hatching rate was 0% until 72 hours, and 8% at 80 hours. It was confirmed that the CP002 treated group (5 μM, 10 μM) had a hatching rate of about 10 to 11% at 60 hours and 100% at 72 hours. In the CP002S 5 μM treated group, the hatching rate was maintained 10% at 48 hours and 20% at 52 hours, and then all hatched (100%) at 72 hours. The CP002S 10 μM treated group had a hatching rate of 80% at 48 hours, and all hatched (100%) at 52 hours. In a DPA 5 μM treated group used as a positive control drug, the hatching rate was 45.4% at 80 hours, and in a DPA 10 μM treated group, the hatching rate was 36.3% at 72 hours and 81.8% at 80 hours. It was confirmed that another positive control group, a TETA 5 μM treated group, had a hatching rate of 12.5% at 48 hours and 75% at 52 hours, and all hatched (100%) at 56 hours. In addition, a TETA 10 μM treated group had a hatching rate of 30.7% at 48 hours, 84.6% at 52 hours, and 92.3% at 72 hours, and all hatched (100%) at 80 hours.

In other words, compared with the normal group, the low hatching rate of 8% at 80 hours under copper overexposure conditions was recovered to 100% at 72 hours in the CP002 treated group at both concentrations, and in the CP002S treated group, the hatching rate was recovered to 80% at 48 hours and 100% at 52 hours within a short time at a concentration of 10 μM rather than 5 μM. The hating rate of the DPA 10 μM treated group was recovered to 36% at 72 hours, which was later than the CP002 and CP002S treated groups. It was confirmed that the hatching rate of the TETA 5 μM treated group was recovered from 12.5% at 48 hours to 100% at 56 hours, which was earlier than that of the CP002 treated group, but the recovery rate was similar to that of the CP002S treated group.

As a result, it was confirmed that the hatching rate delayed by the copper excess capacity was recovered by the CP002S treatment, which means that the CP002S has an excellent effect of inhibiting the action of excess copper in the body.

### Example 8. Therapeutic Effect of CP002S Peptide on Myelination Defect of in Nervous System in Copper Overexposure Nervous System Model

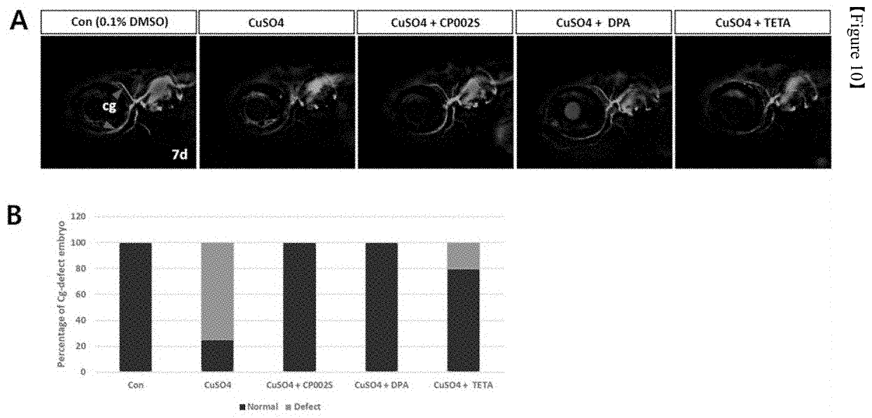

The development process of fertilized eggs obtained by mating male and female wild-type zebrafish was safely maintained in an incubator (28.5° C.). At 24 hours after fertilization, normally grown embryos were dispensed into a 24-well plate by 10 subjects and maintained under copper overexposure conditions. To examine the developmental defects of myelinated cells in the nervous system due to copper overexposure and a therapeutic effect of a multi-copper oxidase peptide, CP002S (10 μM) was treated. After 6 days of drug treatment, the subjects were anesthetized with tricaine and placed in 3% methylcellulose. The myelination defect of cranial ganglia (Cg) was observed through fluorescence microscopy, and fluorescent proteins were divided into a normal and a defect according to an expression level. A negative control group in the experiment was a 0.1% DMSO treated group (normal group) and a copper overexposed group, and a positive control group was a DPA (penicillamine/D-Penicillamine) treated group; and a TETA (trientine/Trientine) treated group. The results observed with a fluorescence microscope were illustrated in FIG. 10A, and the results of analyzing the therapeutic effect based on the fluorescence microscope observation results were illustrated in FIG. 10B.

As illustrated in FIGS. 10A and 10B, it was confirmed that in the normal group (0.1% DMSO), all subjects were normal in the cranial ganglia. However, it was confirmed that the fluorescent protein expression in the cranial ganglia was reduced by 75% in the copper overexposed (4 μM CuSO₄) group. When 10 μM of CP002S was treated with copper, the expression of the fluorescent protein was observed in the cranial ganglia of all subjects. As a result of treating DPA or TETA with copper, it was confirmed that subjects expressing the fluorescent protein were 100% and 80%, respectively. The result means that CP002S has a therapeutic effect on the myelination damage in the nervous system caused by copper excess, like DPA and TETA, which are marketed as therapeutic agents in the copper overexposure model.

### Example 9. Identification of Cardiotoxicity of CP002 and CP002S Peptides in Copper-Overexposed Nervous System Model

The development process of fertilized eggs obtained by mating male and female wild-type zebrafish was safely maintained in an incubator (28.5° C.). At 24 hours after fertilization, normally grown embryos were dispensed into a 24-well plate by 20 subjects and maintained under copper overexposure conditions. On 4.5 days after treatment (5.5 days after fertilization), 7 subjects per group were randomly selected, 3% methylcellulose was put in a hole slide glass, and the heart of each subject was located on the hole slide glass to be observed with the naked eye through a stereoscopic microscope lens and observed under a microscope. The heart rate was measured together with the movement of the atria and the ventricles for 20 seconds for each subject in each group to be converted to a heart rate per minute and the average heart rate for each group was compared through a PRISM (Bonfferroni's Multiple Comparison Test, One-way ANOVA) program. In addition, the heart rate inhibition rate was calculated as follows using an average heart rate of the control group.

Heart rate inhibition rate=[(average heart rate by group−average heart rate in control group)×100]/average heart rate in control group

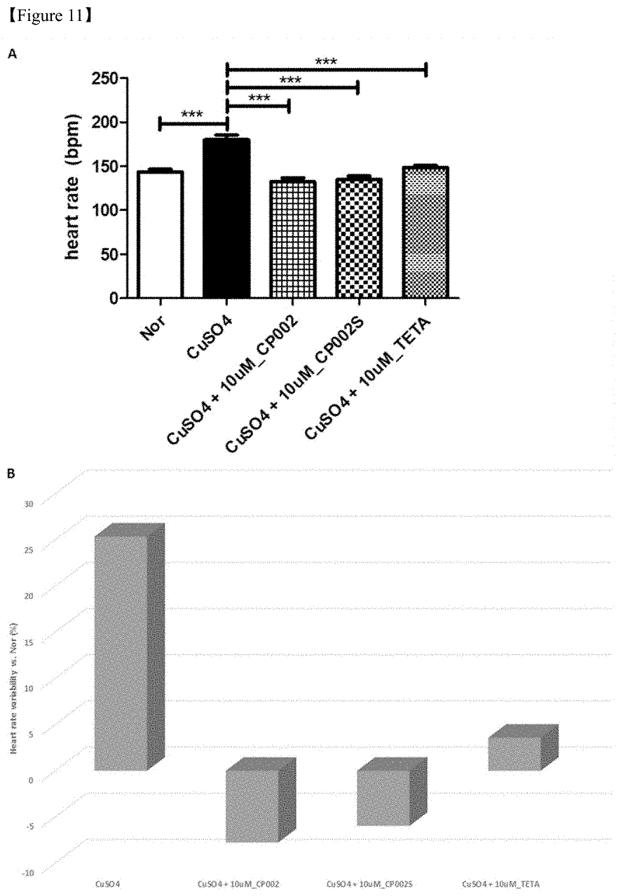

A negative control group in the experiment was a 0.1% DMSO treated group (normal group) and a copper overexposed group (4 μM CuSO₄), and a positive control group was a CP002 treated group; and a TETA (trientine/Trientine) treated group. The results of measuring the heart rate in the copper overexposed model animal were illustrated in FIG. 11A, and the heart rate inhibition rate calculated based on the heart rate measurement results was illustrated in FIG. 11B.

As illustrated in FIGS. 11A and 11B, it was confirmed that in a normal group (nor), the heart rate was 144 beats per minute on 5.5 days after fertilization. It was confirmed that the heart rate of the copper overexposed group increased by 25% to 180 beats per minute. It was confirmed that the CP002S 10 μM treated group had a heart rate of 135 beats per minute, which was decreased by about 6% compared to the normal group. It was confirmed that in a positive control group, the CP002 treated group, the heart rate decreased by 7.7% (132 beats per minute) compared to the normal group, and in the TETA treatment group, the heart rate increased by 3.5% (148 beats per minute) compared to the control group. As the result, it was confirmed that the abnormal increase in heart rate due to copper overexposure was recovered in TETA-treated subjects used as a copper excretion promoter, and also recovered to a normal range even in subjects treated with CP002 and CP002S.

As described above, specific parts of the present invention have been described in detail, and it will be apparent to those skilled in the art that these specific techniques are merely preferred embodiments, and the scope of the present invention is not limited thereto. Therefore, the substantial scope of the present invention will be defined by the appended claims and their equivalents.

1-14. (canceled)

15. A peptide represented by an amino acid sequence shown in SEQ ID NO: 3, 4 or 7.

16. The peptide of claim 15, wherein the peptide is derived from ceruloplasmin.

17. The peptide of claim 16, wherein the ceruloplasmin is derived from Octopus minor.

18. A method for preventing, alleviating or treating neurological diseases comprising:
administering a composition comprising one or more peptides selected from the group consisting of amino acid sequences shown in SEQ ID NOs: 3, 4, and 7 to a subject in need thereof.

19. The method for preventing, alleviating or treating neurological diseases of claim 18, wherein the composition is a pharmaceutical composition, food composition or health functional food composition.

20. The method for preventing, alleviating or treating neurological diseases of claim 18, the concentration of the peptide is 0.01 to 20 μM.

21. The method for preventing, alleviating or treating neurological diseases of claim 18, wherein the neurological diseases are demyelinating neurological diseases.

22. The method for preventing, alleviating or treating neurological diseases of claim 21, wherein the demyelinating neurological diseases are one or more selected from the group consisting of multiple sclerosis, ophthalmoneuromyelitis, and acute demyelinating encephalomyelitis.

23. The method for preventing, alleviating or treating neurological diseases of claim 18, wherein the neurological diseases are neurological diseases induced by copper metabolism disorders.

24. The method for preventing, alleviating or treating neurological diseases of claim 23, wherein the neurological diseases are one or more selected from the group consisting of Parkinson's disease, Alzheimer's disease, amyotrophic lateral sclerosis, Huntington's disease, Menkes disease, Wilson's disease, Prion's disease, occipital horn syndrome, Aceruloplasminemia, abnormal sensory symptoms, movement disorders, and optic neuritis.
